(19) United States
(12) Patent Application Publication
(10) Pub. No.: US 2022/0051761 A1
(43) Pub. Date: Feb. 17, 2022

(54) Process for Research of Causal Links to Lower MSK Injuries-stress fractures

(71) Applicant: Lori Lewis, Groton, MA (US)

(72) Inventor: Lori Lewis, Groton, MA (US)

(21) Appl. No.: 16/850,178

(22) Filed: Aug. 12, 2020

(51) Int. Cl. G16H 10/20; A61B 5/00; G16H 50/50; G16H 50/30; G16H 50/70; G16H 70/20; G09B 19/00

(52) U.S. Cl. CPC G16H 10/20; A61B 5/4509; A61B 5/7275; G16H 50/50; G16H 50/30; A61B 5/4523; G16H 70/20; G09B 19/00; A61B 2503/42; A61B 5/4533; G16H 50/70

(57) ABSTRACT

The invention is measurement of two categories of medical attributes that proceed the causes previously measured to cause lower musculoskeletal (MSK) injuries including stress fractures. The innovation is the measurement of increase in the risk for lower musculoskeletal (MSK) injuries, including stress fractures, due to genetic factors that cause disease (pre-cursors of the disease) as an independent cause for an increase in the risk of MSK injuries, and the diseased states themselves as a cause of increased risk for lower MSK injuries. The method to be used for this measurement will be through statistical and quasi-experimental design. The innovation of measuring the genetic pre-cursor of diseases and these diseases themselves includes them as antecedents for the previously measured causes of stress fractures; such as biomechanical and anatomical factors; muscle power; muscle, ligament and tendon flexibility; equipment used; nutritional; bone mineral density; smoking; amenorrhea; estrogen, among others.

### CROSS-REFERENCE TO RELATED APPLICATIONS

Not Applicable

### STATEMENT REGARDING FEDERALLY SPONSORED RESEARCH OR

### DEVELOPMENTS

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

The field of the invention involves the process to be used in medical research on the causes of stress fractures.

The invention is the method to be used to measure, via statistical and quasi-experimental design, the increase in the risk for lower musculoskeletal (MSK) injuries, including stress fractures, due to genetic factors that cause disease (pre-cursors of the disease) as an independent cause for an increase in the risk of MSK injuries, and the diseased states themselves as a cause of increased risk for lower MSK injuries.

The innovation here are pre-cursors to causes of stress fractures previously. As precursors to the here-to-fore studied causes, this measurement is to provide an earlier warning system for the risks. In terms of statistical regression-based research many of the links studied to-date are no longer independent variables but become intermediate and interactive variables linking to lower MSK injuries. In terms of logic models the factors studied previously are now measured as outputs from the disease precursors and disease states which then produce the outcome of lower MSK injuries to include stress fractures.

Stress fractures are most common from running, dancing and military activities. Stress fractures occur more often in females than males. The genetic factors and subsequent disease states vary greatly by gender. The rate of stress fractures in the U.S. military are three times higher for females than for males. (Studies on military stress fractures in female soldiers are also available regarding military/armies of Australia, Brazil, Israel, Finland, and the United Kingdom.) Measuring the extent to which these genetic and disease affect stress fractures through prior measured factors (that would now be intervening factors given this innovation regarding antecedents) are important to understanding the medical contributors to stress fractures and the possible interventions to lower these risks.

### BRIEF SUMMARY OF THE INVENTION

The invention is measurement of two categories of medical attributes that proceed the causes previously measured to cause by linkages to lower musculoskeletal (MSK) injuries including stress fractures. The innovation is the measurement of increase in the risk for lower musculoskeletal (MSK) injuries, including stress fractures, due to genetic factors that cause disease (pre-cursors of the disease) as an independent cause for an increase in the risk of MSK injuries, and the diseased states themselves as a cause of increased risk for lower MSK injuries. The method to be used for this measurement will be through statistical and quasi-experimental design. The innovation of measuring genetic pre-cursor of diseases and these diseases themselves includes them as antecedents for the previously measured causes of stress fractures; such as biomechanical and anatomical factors; muscle power; muscle, ligament and tendon flexibility; equipment used; nutritional; bone mineral density; smoking; amenorrhea; estrogen, among others.

### BRIEF DESCRIPTION OF THE SEVERAL VIEWS OF THE DRAWING

Not Applicable

### DETAILED DESCRIPTION OF THE INVENTION

The invention is measurement of two categories of medical attributes that proceed the causes previously measured to cause by linkages to lower musculoskeletal (MSK) injuries including stress fractures. The innovation is the measurement of increase in the risk for lower musculoskeletal (MSK) injuries, including stress fractures, due to genetic factors that cause disease (pre-cursors of the disease) as an independent cause for an increase in the risk of MSK injuries, and the diseased states themselves as a cause of increased risk for lower MSK injuries. The method to be used for this measurement will be through statistical and quasi-experimental design.

The innovation of measuring genetic pre-cursor of diseases and these diseases themselves includes them is as antecedents for the previously measured causes of stress fractures. Prior work regarding research of the causes of stress fractures include: biomechanical and anatomical factors; muscle power; muscle, ligament and tendon flexibility; equipment used; nutritional; bone mineral density; smoking; amenorrhea; estrogen, among others. Literature search of research articles found no studies measuring and none identifying antecedents to the causes previously researched, as cited above.

A handful of the many research literature of prior work is included here. These each have long lists of cites.

Kimberley A. Andersen, Paul N. Grimshaw, Richard M. Kelso (School of Mechanical Engineering, University of Adelaide, Adelaide, South Australia 5005 Australia) and David J. Bentley (School of Health Sciences, Flinders University, GPO Box 2100, Adelaide, South Australia 5001 Australia)(2016) “Musculoskeletal Lower Limb Injury Risk in Army Populations”, Sports Medicine—Open, December 2016, Volume 2: 22.

Army Public Health Fact Sheet 12-021-0319—Preventing Injuries in Female Soldiers

Diego Costa Astur, Fernando Zanatta, Gustavo Gonsalves Arliani, Eduardo Ramalho Moraes, Alberto de Castro Pochini, and Benno Ejnisman (Work performed at the Sports Traumatology Center, Escola Paulista de Medicina, Universidade Federal de São Paulo, São Paulo, SP, Brazil) (2016) “Stress fractures: definition, diagnosis and treatment”, Rev Bras Ortop (Revista Brasileira de Ortopedia, scientific publication of Brazilian Society of Orthoapedics and Traumatology), January-February 2016, Volume51, Issue 1, pages 3-10.

Yin-Ting Chen, Adam S. Tenforde, and Michael Fredericson (Department of Orthopaedic Surgery, Division of Physical Medicine and Rehabilitation, Stanford University, Redwood City, Calif.)(2013) “Update on stress fractures in female athletes: epidemiology, treatment, and prevention”, Current Reviews in Musculoskeletal Medicine (Curr Rev Musculoskelet Med.) June 2013, Volume 6, Issue 2, pages 173-181.

Karl E. Friedl, MSC USA, Jennifer A. Nuovo, MD, Troy H. Patience, DAC, Joseph R. Dettori, AMSC (1992) “Factors Associated with Stress Fracture in Young Army Women: Indications for Further Research”, Military Medicine, Volume 157, Issue 7, July 1992, Pages 334-338.

Mattila, Ville M, Niva, Maria and Kiuru, Martti (Centre for Military Medicine, Lahti, Finland), and Pihlajamaki, Harri (Department of Medical Services, Defence Staff, Finnish Defence Forces, Helsinki, Finland)(2007) “Risk Factors for Bone Stress Injuries: A Follow-up Study of 102,515 Person-Years”, Medicine & Science in Sports & Exercise, July 2007-Volume 39-Issue 7-pages 1061-1066.

Nkechinyere Chidi-Ogbolu (Biomedical Engineering Graduate Group, University of California, Davis, Davis, Calif., U.S.) and Keith Baar (Departments of Neurobiology, Physiology and Behavior, and of Physiology and Membrane Biology, University of California, Davis, Davis, Calif., U.S.)(2018). “Effect of Estrogen on Musculoskeletal Performance and Injury Risk”, Frontiers of Physiology. 2018 Volume 9, Pages 1834-1863.

Oren Schwartz, MD IDF Medical forces, ltzik Malka, MD IDF Medical forces, Cara H Olsen, Prof, Israel Dudkiewicz, Prof , Tarif Bader, MD IDF Medical forces (2018) “Overuse Injuries Among Female Combat Warriors in the Israeli Defense Forces: A Cross-sectional Study”, Military Medicine, Volume 183, Issue 11-12, November-December 2018, Pages e610-e616.

Laurel Wentz, MS, RD, Pei-Yang Liu, PhD, RD, Emily Haymes, PhD, Jasminka Z. Ilich, PhD, RD, (2011) “Females Have a Greater Incidence of Stress Fractures Than Males in Both Military and Athletic Populations: A Systemic Review”, Military Medicine, Volume 176, Issue 4, April 2011, Pages 420-430.

1. A method of scientific research measuring the influence of variables causing or correlating to musculoskeletal (MSK) injuries comprising a multiple of physical attributes; a multiple of health status; a multiple of diseases; a multiple of genetic precursors to diseases.
A. The method of claim 1 wherein the physical attributes are increasing the likelihood of physical changes comprising subsequent physical attributes; changes in medical physical abilities; changes in medical processes.
B. The method of claim 1 wherein multiple diseases increasing the likelihood of health and nutrition states.
C. The method of claim 1 wherein precursors to diseases increasing the likelihood of health and nutrition states.

2. A method of research design for creating likelihood measurements for claim 1 comprising identification of research questions; identifying datasets available for research use;
documenting prior research for specifications of statistical models being used in research design; identifying component research to be used within the process model in claim 1;
identify and creating datasets for research analysis; conducting statistical modeling to measure likelihoods of elements in claim 1; conduct statistical modeling measuring likelihoods comprising interactions between variables in all the above claims.
